(19) United States
(12) Patent Application Publication
(10) Pub. No.: US 2010/0291000 A1
(43) Pub. Date: Nov. 18, 2010

(54) Diagnostic value of systemic ACE pathway activation in the detection by fluorescence of localized pathological lesions

(76) Inventor: William John Martin, South Pasadena, CA (US)

Correspondence Address: William John Martin, 1634 Spruce Street, South Pasadena, CA, 91030, US

(21) Appl. No.: 12/804,619

(22) Filed: Jul. 26, 2010

Related U.S. Application Data
(63) Continuation of application No. 12/069,597, filed on Feb. 11, 2008.

(51) Int. Cl. A61K 49/00

(52) U.S. Cl. 424/9.6

(57) ABSTRACT

Alternative cellular energy pigments (ACE-pigments) provide a source of cellular energy other than that provided through the oxidative metabolism of foods, or in the case of plants and certain bacteria, through the process of photosynthesis. In some patients, ACE pigments exist in a form that can be further energized or activated using ultraviolet (UV) light, especially if the reaction is initially triggered by the presence of suitable dyes, such as neutral red. Once partially activated, ACE pigments within a localized lesion will fluoresce upon direct exposure to UV light. It is diagnostically useful to screen an individual or animal undergoing localized or systemic activation of their ACE pathway for the presence of lesions elsewhere in the body, which become fluorescent under direct UV illumination, both as a way of detecting an otherwise non-apparent or unnoticed lesion and of confirming that such a lesion contains ACE pigments that can be further activated. The development of direct UV fluorescence in a localized lesion can also be an indication of the therapeutic value of continuing with direct UV illumination of the lesion.

### CROSS REFERENCE TO RELATED APPLICATIONS

### Co-Pending Patent Application (and others)

Method of Assessing and of Activating the Alternative Cellular Energy (ACE) Pathway in the Therapy of Diseases. William John Martin Submitted Jun. 8, 2010

“Method of Using the Body's Alternative Cellular Energy Pigments (ACE-pigments) in the Therapy of Diseases.” William John Martin Submitted Feb. 20, 2009

Enerceutical Mediated Activation of the Alternative Cellular Energy (ACE) Pathway in the Therapy of Diseases. 12/069,597 William John Martin. Submitted Feb. 11, 2008

Method of Assessing and of Activating the Alternative Cellular Energy (ACE) Pathway in the Therapy of Diseases. William John Martin. Submitted Jan. 17, 2008

### Previously Submitted but Now Abandoned Patent Applications

10/044,683. Therapy of stealth virus associated cancers and other conditions using light. William John Martin. (Abandoned)

10/047,313. Therapy of stealth virus associated cancers and other conditions using medium chain triglycerides. William John Martin. (Abandoned)

10/050,232. Diagnosing and monitoring the therapy of stealth virus infections based on the detection of auto-fluorescent material in hair. William John Martin. (Abandoned)

10/058,480. Therapy of stealth virus associated cancers and other conditions using magnetic energy. William John Martin. (Abandoned)

10/164,258 Energy supportive therapy of stealth virus associated diseases. William John Martin. (Abandoned)

10/174,466 Sound therapy of stealth virus associated diseases. William John Martin. (Abandoned)

10/192,936 ACE-Pigments and humic acids as energy sources. William John Martin. (Abandoned)

Submitted: 10/ Methods for Collection of Alternative Cellular Energy Pigments (ACE-pigments). William John Martin. (Abandoned)

Submitted: 10/ Methods for Elimination of Toxic Alternative Cellular Energy Pigments (ACE-pigments) and for Their Replacement Using Activated Humates, Including Humic and Fulvic Acids. William John Martin. (Abandoned)

### United States Patents (Awarded)

U.S. Pat. No. 5,985,546 Stealth virus detection in the chronic fatigue syndrome. William John Martin

U.S. Pat. No. 5,891,468 Stealth virus detection in the chronic fatigue syndrome. William John Martin

U.S. Pat. No. 5,753,488 Isolated stealth viruses and related vaccines. William John Martin

U.S. Pat. No. 5,703,221 Stealth virus nucleic acids and related methods. William John Martin

### PCT (Patent Cooperation Treaty)

WO 92/20797 Stealth virus detection in the chronic fatigue syndrome. William John Martin

WO 99/34019 Stealth virus nucleic acids and related methods. William John Martin

WO 99/60101 Stealth viruses and related vaccines. William John Martin

### REFERENCES TO PUBLISHED ARTICLES

### Alternative Cellular Energy Pigments (ACE-pigments)

1 Martin WJ. Alternative cellular energy pigments mistaken for parasitic skin infestations. Exp. Mol. Path 78: 212-214, 2005.

2 Martin WJ. Alternative cellular energy pigments from bacteria of stealth virus infected individuals. Exp. Mol. Path 78: 217-217, 2005.

3 Martin WJ. Progressive Medicine. Exp Mol Path 78: 218-220, 2005.

4 Martin WJ, Stoneburner J. Symptomatic relief of herpetic skin lesions utilizing an energy based approach to healing. Exp. Mol. Path 78: 131-4, 2005.

5 Martin WJ. Etheric Biology. Exp Mol Path 78: 221-227, 2005.

6 Martin WJ. Stealth Virus Culture Pigments: A Potential Source of Cellular Energy. Exp. Mol. Pathol. 74: 210-223, 2003.

7 Martin WJ. Complex intracellular inclusions in the brain of a child with a stealth virus encephalopathy. Exp. Mol. Pathol. 74: 179-209, 2003.

8 Martin WJ. Photons and phonons: Theoretical aspects of biophysics and potential therapeutic applications. Proceeding of Neural Therapy Workshop on Sound and Light Therapy, Seattle, WA, Feb. 21-23, 2003.

### Stealth Adapted Viruses

1 Martin WJ Chronic fatigue syndrome among physicians. A potential result of occupational exposure to stealth viruses. Explore 2001; 10: 7-10.

2 Martin WJ. Stealth Viruses. Explore 2001; 10: 17-19.

3 Dune GM, Collins R. Martin WJ. Positive stealth virus cultures in multiple myeloma. A possible explanation for neuropsychiatric co-morbidity. Presented at the Am. Soc. Hematology annual meeting October 2000.

4 Martin WJ. Chemokine receptor-related genetic sequences in an African green monkey simian cytomegalovirus-derived stealth virus. Exp Mol Pathol. 2000; 69:10-6.

5 Martin WJ., Anderson D. Stealth virus epidemic in the Mohave Valley: severe vacuolating encephalopathy in a child presenting with a behavioral disorder. Exp Mol Pathol. 1999; 66:19-30.

6 Martin WJ. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin WJ. Bacteria-related sequences in a simian cytomegalovirus-derived stealth virus culture. Exp Mol Pathol. 1999; 66:8-14.

8 Martin WJ. Stealth adaptation of an African green monkey simian cytomegalovirus. Exp Mol Pathol. 1999; 66:3-7.

9 Martin WJ. Cellular sequences in stealth viruses. Pathobiology 1998; 66:53-8.

10 Martin WJ. Detection of RNA sequences in cultures of a stealth virus isolated from the cerebrospinal fluid of a health care worker with chronic fatigue syndrome. Case report. Pathobiology. 1997; 65:57-60.

11 Martin WJ., Anderson D. Stealth virus epidemic in the Mohave Valley. I. Initial report of virus isolation. Pathobiology. 1997; 65:51-6.

12 Martin WJ. Simian cytomegalovirus-related stealth virus isolated from the cerebrospinal fluid of a patient with bipolar psychosis and acute encephalopathy. Pathobiology. 1996; 64:64-6.

13 Martin WJ. Stealth viral encephalopathy: report of a fatal case complicated by cerebral vasculitis. Pathobiology. 1996; 64:59-63.

14 Martin WJ. Genetic instability and fragmentation of a stealth viral genome. Pathobiology. 1996; 64:9-17.

15 Martin WJ. Severe stealth virus encephalopathy following chronic-fatigue-syndrome-like illness: clinical and histopathological features. Pathobiology. 1996; 64:1-8.

16 Martin WJ. Stealth virus isolated from an autistic child. J Autism Dev Disord. 1995; 25:223-4.

17 Gollard RP, Mayr A., Rice DA, Martin WJ. Herpesvirus-related sequences in salivary gland tumors. J Exp Clin Cancer Res., 1996; 15: 1-4.

18 Martin WJ., Glass RT. Acute encephalopathy induced in cats with a stealth virus isolated from a patient with chronic fatigue syndrome. Pathobiology. 1995; 63:115-8.

19 Martin WJ, et al. African green monkey origin of the atypical cytopathic ‘stealth virus’ isolated from a patient with chronic fatigue syndrome. Clin Diag Virol 1995: 4: 93-103.

20 Martin WJ. Stealth viruses as neuropathogens. CAP Today. 1994; 8: 67-70.

21 Martin WJ. et al. Cytomegalovirus-related sequence in an atypical cytopathic virus repeatedly isolated from a patient with chronic fatigue syndrome. Am J Pathol. 1994; 145: 440-51.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX.

Not applicable.

### BACKGROUND OF THE INVENTION:

The invention is based on the following broad conceptual understanding on how the body can acquire cellular energy other than through the oxidative metabolism of foods. Essentially, an alternative cellular energy (ACE) pathway has been identified that is mediated by the energy converting (transducing) properties of complexes of organic molecules, arbitrarily termed alternative cellular energy pigments (ACE pigments). Cellular energy generated by this pathway can seemingly complement the chemical energy that is derived by living organisms from the metabolism of food. The ACE pathway is likely to contribute to various physiological functions of the body. Of particular relevance to this application, the ACE pathway is postulated to provide an auxiliary defense mechanism beyond that of the immunological system, such that an inadequacy of this pathway may limit the body's healing and regenerative capacities in response to infectious and other disease processes. The ACE pathway is also anticipated to be involved in the normal functioning of many organs, including the brain. Moreover, it is reasonable to presume that many illnesses, not necessarily of infectious origin, may place an added burden on the ACE pathway and that an inadequacy or deprivation of the ACE pathway may be a factor in delaying the normal disease recovery process. Conversely, augmenting or activation of an impaired ACE pathway may facilitate recovery from a wide range of various illnesses. Moreover, a fully functioning ACE pathway is likely to also be a factor in maintaining wellness, enhancing athletic performance, increasing cognitive abilities, etc.

The body's ACE pigments are envisioned as tiny batteries with differing levels of being “charged.” The working model postulates that uncharged or very poorly charged ACE pigments will not fluoresce when illuminated directly with an ultraviolet light, (for example a 13 watt Halco spiral UV light bulb or a Streamlight 365 nm LED stylus penlight.) They will, however, fluoresce when mixed with certain dyes, including neutral red (e.g. at 0.1 mg/ml; available from Dudley Chemicals, New Jersey). ACE pigments can also alter the fluorescence pattern of other dyes, including acridine orange and Stains-All. Once partially charged, ACE pigments will fluoresce directly when illuminated with UV light. The UV illumination is able to provide additional energy to the ACE pigments and when fully charged, the ACE pigments will, seemingly, not absorb further energy and, therefore, will longer fluoresce under UV illumination, in either the presence or absence of neutral red dye. The three energy levels classification of ACE pigments has proven useful in the clinical assessment of patients. ACE pigments can be collected from areas of skin and skin lesions, hair, bodily fluids, including perspiration, saliva, urine and blood. In blood, the ACE pigments can be seen both within cells, especially in neutrophils and in serum/plasma. A simple home-based fluorescence screening method is for people to collect material onto a gauze swab or Q-tip and to test fluorescence before and after the addition of neutral red dye. Note that some, but not all, gauze swabs and Q-tips can cause a low level of neutral red fluorescence, especially at alkaline pH. It is, therefore, important to select an appropriate sampling material, which can include wooden spatulas, laboratory filter paper and some brands of coffee filter papers. Alternatively, saliva and urine can be collected in a UV transparent container, such as disposable plastic glass, and examined before and after the addition of neutral red for UV fluorescence. Alcohol can be used as a positive control since it will render neutral red fluorescence, especially if the solution is slightly alkaline. Neutral red dissolved in water provides a negative control. Separately from testing material taken from the subject, it is also informative to examine parts of the body under direct UV illumination. Thus, any areas of direct UV fluorescence provides a tentative indication for the presence of partially charged ACE pigments. It can typically occur within the oral cavity, diffuse areas of skin and, as disclosed in this application, in localized pathological lesions that are present elsewhere on or within an individual.

As disclosed in this patent application, systemic (that is, whole body) ACE pathway activation can, therefore, be used diagnostically in the detection of lesions that might otherwise be go undetected. Thus, areas of the skin and other surface areas of the body can be screened for UV fluorescence, both before and after systemic activation of the ACE pathway. Any area of fluorescence can be tentatively identified as a pathological lesion at which the body is apparently attempting to utilize the ACE pathway for repair.

### BRIEF SUMMARY OF THE INVENTION

Systemic ACE pathway activation in an ACE deficient individual can be achieved by several methods; including placing an ultraviolet illuminated solution of neutral red that is dissolved in ethanol onto the body. Preferred site for activation are areas in which there are chronic skin lesions and on highly innervated areas, such as the palms, soles of the feet, face and neck. In an ACE pathway deficient individual, this procedure can evoke oral fluorescence upon direct UV illumination of the mouth. It can also evoke the direct UV fluorescence of other types of underlying skin lesions, beyond those caused by herpes infections. The appearance of fluorescence in these lesions can be diagnostically useful in the detection of an unsuspected lesion, screening for possible sub-clinical herpes virus outbreaks, and for confirmation that the body is attempting to use its ACE pathway in the healing of the lesion.

### BRIEF DESCRIPTION OF THE DRAWINGS

Not Applicable and none included.

### DETAILED DESCRIPTION OF THE INVENTION

An incidental observation in patients undergoing systemic ACE pathway activation has been reports of localized lesions of the skin becoming fluorescent if they are directly exposed to UV light. The localized fluorescence is distinguished from the diffuse fluorescence that can occasionally be seen within skin areas, e.g. surrounding a skin lesion, palms, soles of the feet and oral cavity. Instead, the localized fluorescence is more intense and confined to one or more skin lesions, which had not been covered with neutral red dye. This phenomenon has been seen in patients with shingles, in whom neutral red had been applied to only some of the lesions; yet all of the lesions displayed on orange to red fluorescence. Similarly, with Morgellon's disease (a condition in which abundant ACE pigments are present in many ulcerating lesions on the body), a patient reported that ulcerated areas of his arms would fluoresce, even though he had only applied neutral red, under my direction, to lesions elsewhere on his body before illuminating much of his body with UV fluorescent light bulbs. A patient with psoriasis also noted induced fluorescence in skin lesions beyond the area on which he applied neutral red.

I have long recognized that this approach can potentially reveal other types of lesions that may not have been previously noted by the patient. For example, a woman noted that while she was treating her HSV lip lesion, a localized area of her thumb began to show slight fluorescence. The fluorescing area on her thumb was quite possibly a sub-clinical herpetic whitlow (HSV infection of the finger). I also realized that the secondarily induced direct fluorescence may also occur with skin or internal bodily lesions, which are pathologically unrelated to the type of lesion being treated. For example, a woman described seeing fluorescence developing in a seborrheic keratosis on her nose while she was treating herself for genital HSV. Typically, the secondary fluorescence is not as marked as the bright yellow fluorescence commonly seen with oral herpes. It is usually a more multicolored yellow-orange-reddish hue, likened to the shading of a fresh peach. The colors are significantly different from simple reflection of the UV light and are clearly emanating from the lesion. It is still visible using a filter that selectively transmits only the UV light component of light source being used. The Streamlight 365 nm stylus light is useful for detailed study of a particular skin lesion.

People with evidence of a deficient ACE pathway are, therefore, being encouraged to look for localized fluorescing skin lesions before, during and immediately after efforts to achieve systemic activation of their ACE pathway. The definition of induced fluorescence is actual light coming from a lesion, which was not observable, or markedly less apparent, prior to beginning the systemic ACE pathway activation process. To help ensure that the colors are not merely a reflection of a visible blue component of light that accompanies some of the UV light sources being used, the visible component of can be blocked using an appropriate filter that selectively transmits UV light.

In a preferred embodiment, UV light illuminated neutral red dissolved in ethyl alcohol can be used to systemically activate the body's own ACE pigments. Approximately 1-5 mg of neutral red freshly dissolved in 10 ml of ethyl alcohol and placed in a sealable plastic bag with or without a small sheet of absorbent paper to help evenly distribute the fluid. The plastic bag is placed onto the surface of the body to ensure direct contact, using tape if necessary. The bag is illuminated with a 13 watt Halco ProLume spiral UV light bulb, or a more powerful BLB fluorescent tubes, which is positioned closely to the solution, such that it will show very bright fluorescence in a darkened room. The therapy session will typically last from 30-60 minutes. Both before and after the procedure, the skin and mucus membrane accessible surfaces can be surveyed using the same type of UV light, or a more focused UV LED light. This survey is in addition to checking for areas of diffuse skin fluorescence or oral fluorescence, which are indicative of the process of achieving systemic, partial, activation of the ACE pathway.

Rather, in the present embodiment, the patient is instructed to see if any localized intensely fluorescing lesions can be seen anywhere on the body. If available, a filter that selectively transmits only UV light can be placed between a suspected lesion and the UV light source. If the lesion continues to glow, it is recorded as being fluorescent. The localized fluorescence can range from yellow (especially for HSV); orange (especially for HZV) and reddish (especially for HPV) and even purplish-blue. Certain skin cancers are also expected to show fluorescence, as will chronic wounds. This approach compliments and is considered less toxic than having to apply neutral red or other suitable pigments directly onto wide areas of suspected pathology, prior to UV illumination.

In one embodiment, the above procedure would be useful in the examination of genital skin areas for a possible subclinical outbreak of HSV. If there were no areas of localized fluorescence, one might feel reassured that acting shedding of herpes viruses was less likely to be occurring than if a localized area of intense fluorescence was observed. This type of survey might be useful, for example, in an HSV positive pregnant women contemplating vaginal rather than caesarian delivery.

In another embodiment, patients are encouraged to photograph any fluorescing lesions discovered during the procedure and for transmitting the photographs over the interne or displaying them via video using Skype or other VOW services.

Beyond home-care, the above procedure should find utility in the endoscopic examination for fluorescing lesions within the respiratory, gastrointestinal and genitourinary systems and other body cavities. A fine needle with both a source of UV light and a detector of fluorescence can also be envisioned for the examination of solid tumors.

The value of detecting fluorescing localized lesions extends to the realization that ACE pathway activation therapy would be of both diagnostic and therapeutic value. Research studies are indicated to catalogue a wide range of lesions, on the skin and internal surfaces of the body for inducible direct UV fluorescence as a result of systemic activation of the ACE pathway.

The monitoring of induced direct fluorescence in a localized skin lesion can also be used in deciding the potential therapeutic value of further direct UV illumination of the lesion, as opposed to indirect activation achieved with neutral red dye in an appropriate solvent, such as alcohol: or in an aqueous solution when mixed with a source of the body's ACE producing materials (ACE pigments) or other suitable material; which are placed directly or indirectly onto an area of the body prior to being UV illuminated. The ability to directly stimulate a lesion with UV light extends to direct exposure to sunlight.

Although neutral red is considered a suitable dye for activating the ACE pathway, various other dyes will also react with ACE pigments and either become fluorescent or modify their preexisting fluorescence. These include but are not limited to acridine orange and Stains-All. Similarly, the use of ethyl alcohol with neutral red can be replaced with other solvents, including methanol. The dyes can be directly applied to the skin or placed onto paper towels or fabric materials and either placed within a plastic or other UV transparent container or used without a container. Sources of patient derived ACE pigments can include saliva, perspiration, urine and materials removed from localized skin lesions in which ACE pigments have accumulated.

The principles, preferred embodiments and modes of operation of the present invention have been described in the foregoing specification. The invention, which is intended to be protected herein, however, is not to be construed as limited to the particular method of systemically activating the ACE pathway, since this is to be regarded as illustrative of the basic principle of using ACE pathway activation as a diagnostic procedure to help identify pathological lesions. The basic claim is, therefore, broader issue of diagnostic benefit through systemic activation of the ACE pathway. Additional advantages and modifications of the basic tenets disclosed in the present patent application will readily occur to those skilled in the art and especially upon practicing the currently described methods. Many variations and changes may be made without departing from the scope and spirit of the invention as encompassed by the appended claims.

WHAT I CLAIM AS MY INVENTION IS:

1. A diagnostic method for detecting and categorizing a pathological lesion in an animal or human subject, based upon observing the lesion becoming directly fluorescent under ultraviolet (UV) light illumination, during or following the process of systemically activating the alternative cellular energy (ACE) pathway in the subject; the actual method comprising the use of a fluorescent light to view external and/or internal surfaces of the body for localized areas of fluorescence, which may range from yellow through orange to red and which develop during the process of systemically activating the ACE pathway, which may be achieved by using UV illuminated neutral red dye in an appropriate solvent, such as alcohol: or in an aqueous solution when mixed with a source of the body's ACE producing materials (ACE pigments) or other suitable materials; with the combined reagents being placed directly or indirectly onto an area of the body prior to being UV illuminated.

2. A method of accessing whether ACE pigments, with the potential of being further activated, exists within a localized skin lesion on a human or animal subject by determining if the lesion will become directly fluorescent when illuminated with an ultraviolet (UV) light following the systemic activation of the alternative cellular energy (ACE) pathway, which is achieved by using UV illuminated neutral red dye in an appropriate solvent, such as alcohol: or in an aqueous solution when mixed with a source of the body's ACE producing materials (ACE pigments) or other suitable material, which are placed directly or indirectly onto an area of the body prior to being UV illuminated; in that fluorescence developing in the localized lesion using the described method is an indication that ACE pigments, with the capacity of being further activated, were indeed present in the localized lesion.

3. A method of predicting the therapeutic value of treating a localized lesion by either the local or systemic activation of the alternative cellular energy (ACE) pathway, by determining whether the lesion will become directly fluorescent when illuminated with an ultraviolet (UV) light following the local or systemic activation of the alternative cellular energy (ACE) pathway, which is achieved by using UV illuminated neutral red dye in an appropriate solvent, such as alcohol: or in an aqueous solution when mixed with a source of the body's ACE producing materials (ACE pigments) or other suitable material, which are placed directly or indirectly onto the skin lesion (local activation) or onto other areas of the body (systemic activation) prior to being UV illuminated; in that fluorescence developing in the localized lesion using the described method is an indication that ACE pigment activation can be predicted as having a therapeutic value in the healing of the localized lesion.

4. The method of claim 2 to assist in the differentiation of chronic persisting lesions, including areas of acute and recurring herpetic skin lesions (within which ACE pigments are predicted to have been deposited), from more acute lesions, such as from acute trauma (in which ACE pigments are predicted not to have been deposited).

5. The method of claim 3 in which the persistence of induced direct UV fluorescence of a localized skin lesion, achieved by the local or system activation of the ACE pathway is used as a indication for the therapeutic value of continued direct UV illumination of the lesion, as opposed to having to further reactivate the ACE pathway using UV illuminated neutral red dye in an appropriate solvent, such as alcohol: or in an aqueous solution when mixed with a source of the body's ACE producing materials (ACE pigments) or other suitable material, which are placed directly or indirectly onto the localized skin lesion (local activation) or onto other areas of the body (systemic activation) prior to being UV illuminated.

6. The method of claim 1 in which systemic activation of the ACE pathway is achieved by using UV illuminated acridine orange or Stains-All dye in an appropriate solvent, such as alcohol: or in an aqueous solution when mixed with a source of the body's ACE producing materials (ACE pigments) or other suitable material; which can activate the body's ACE pathway when placed directly or indirectly onto an area of the body prior to being UV illuminated.

7. The method of claim 1 in which a kit is provided that contains one or more of the following components: neutral red dye, alcohol solution, ultraviolet light, filter that selectively transmits ultraviolet light, plastic bags and a set of instructions.
